(19) United States
(12) Patent Application Publication
(10) Pub. No.: US 2010/0286770 A1
(43) Pub. Date: Nov. 11, 2010

(54) Support Element for Compensating for Deformations of the Cornea of an Eye

(76) Inventors: Mark Tomalla, Mulheim a. d. Ruhr (DE); Gebhard Tomalla, Essen (DE)

Correspondence Address: Sunstein Kann Murphy & Timbers LLP, 125 SUMMER STREET, BOSTON, MA, 02110-1618, US

(21) Appl. No.: 12/295,494

(22) PCT Filed: Mar. 29, 2007
(86) PCT No.: PCT/EP07/53063

§ 371 Date: Oct. 27, 2009

(30) Foreign Application Priority Data
Mar. 30, 2006 (DE) 10 2006 015 172.0

(51) Int. Cl. A61F 2/14

(52) U.S. Cl. 623/5.12

(57) ABSTRACT

The invention relates to a support element to compensate for deformations of the cornea H of an eye, having an annular support body 2 in one or more pieces, which makes it possible to compensate for keratoconus with minimised stress on the cornea of the eye being treated. This is achieved according to the invention in that the support body 2 has a cross section which is flattened on one side and otherwise rounded, so that a circumferential front-side flattening 6 is formed on it.

The invention relates to a support element to compensate for deformations of the cornea of an eye, which comprises a transversely slit annular support body. Such support elements, sometimes also referred to as support rings here, are inserted into a channel which is made in the cornea of a human or animal eye and in general extends parallel to its contour, in order to remove local protrusions of the cornea which restrict vision.

Such protrusions are also referred to as “keratoconus” in the specialist terminology. They occur owing to progressive thinning and consequent cone-shaped deformation of the cornea of the eye.

It is known that outwardly curved corneal deformations can be compensated for in keratoconus patients by curvatures raised in the opposite direction, i.e. into the interior of the eye. These curvatures are produced by inserting displacement bodies, so-called “intacs”, into the proximal cornea half, i.e. the half facing towards the body, as much as possible outside the field of view or in its peripheral zone.

The known intacs used as support elements are usually designed annularly with a round or hexagonal cross section, and they produce an annular excrescence after implantation in the cornea. Transversely acting tensile forces are produced in the cornea owing to the excrescence formation. These can be adapted by corresponding configuration of the support element itself, as well as of the channel, so that the pathological corneal swelling (keratoconus) is compensated for according to the normal corneal curvature.

In order to be able to be introduced in a simple manner into the channel formed in the cornea of the eye, the known support elements are conventionally composed of two half-rings. Such a ring is described, for example, in EP 0 732 891 B1. In the final implanted state, this ring has a shape resembling a disc spring with conically chamfered front-side bearing surfaces. By means of these surfaces, which in the implanted state bear tightly on the inner surfaces of the channel formed in the cornea, the ring exerts tensile forces on the corneal section enclosed by the support ring, by which the relevant corneal section is tensioned and therefore smoothed.

Based on practical experience with support elements of the type described above, there is a desire for a support element which makes it possible to compensate for keratoconus with minimised stress on the cornea of the eye being treated.

This object has been achieved according to the invention by a support element designed according to claim 1. Advantageous embodiments of this support element are specified in the claims dependent on claim 1.

The invention provides a support element to compensate for deformations of the cornea of an eye, which in a manner known per se has an annular support body in one or more pieces. According to the invention, this support body has a cross section which is flattened on one side. According to the invention, this flattening is arranged so that a circumferential front-side flattening is formed on the support element according to the invention. In the implanted state, this flattening forms a bearing surface with which the support element according to the invention bears tightly on the outwardly directed inner wall of the slit-like channel which has previously been formed, for example by means of a laser beam, in the cornea of the eye respectively being treated. Outside its flattened section, the cross section of the support element according to the invention is rounded. This ensures that the forces exerted by a support element according to the invention are induced gently into the sections of the cornea which enclose the support element and local load peaks are avoided.

A support element according to the invention is therefore shaped so that the detrimental effect on the corneal inner wall, encountered in the prior art due to curvature and strain induced stresses, is kept as small as possible. A support element configured according to the invention is therefore highly appropriate for implantation into a slit-like circle annular channel which is made with the aid of a very high-frequency laser, for example a femtosecond laser, in the cornea of the eye being treated and extends parallel to the corneal contour and therefore to a first approximation conically, and whose shape resembles that of a disc spring.

The orientation of the flattening provided on a support element according to the invention is preferably to be selected so that it is adapted to the orientation of the inner face of the channel slit formed in the eye cornea, which comes in contact with it in the implanted state. The advantages of the embodiment of the flattening adapted to the inclination angle of the contact surface of the channel acting as a support surface in the implanted state, reside in the small surface pressure and the therefore small impression behaviour into the cornea in the region of the flattening.

The flattening provided in a support element according to the invention can consequently be designed so that the forces transmitted by it are adapted optimally to the conditions respectively existing locally in its vicinity after its implantation. To this end, in cases in which the geometry and the state of the corneal section, into which the support element is intended to be introduced, are formed uniformly, the flattening formed on the support element according to the invention may form an annular disc surface tapering in the form of a cone. A support element shaped in this way can be produced particularly simply and inserted precisely into the channel slit formed in the cornea to be treated.

A conically configured flattening furthermore allows a stroma region which is relatively large in relation to the thickness of the cornea, to be selected for implantation of the support ring. A flattening extending almost horizontally, i.e. approximately parallel to the circle plane of the support element, is on the other hand preferably to be restricted to the proximal cornea half in order to avoid or preclude the consequences of the support ring pressing through outwards.

An essential advantage of a support element according to the invention in this case consists in the high centring effect, which is achieved owing to its conically configured flattening in the described corneal channel.

In order to be able to effect a large functional range with the preferably large support surface, the angle setting (flattening angle) relative to the ring plane of the support element of the flattening acting as a support surface is claimed between about 25° to extending beyond the horizontal, here 0° . Via the alignment of the flattening, it is possible to adjust the shear force which is induced in the surrounding cornea by the support element in the implanted state. Depending on the geometry and position of the channel made in the cornea, load peaks can in this case be avoided by the flattening angle being >0° .

Owing to the fact that the flattening, in a horizontal alignment, does not extend parallel to the channel wall produced in the curved cornea, but at an angle to it, the horizontally aligned flattening exerts a shear force component on the cornea's region lying outwards beyond the front-side surface.

The greatest reliability in predicting the operation outcome, and therefore in establishing the dimensions to be selected for a support element according to the invention, is to be achieved in the case of slit channels provided in the proximal corneal region. This arrangement of the channel, to be made for implantation of a support element according to the invention, entails a large thickness of the cornea's region lying outwards relative to the channel slit compared with the thickness of the cornea's region lying inwards relative to the channel slit.

In order to effect uniform induction of the forces introduced by a support element according to the invention into the cornea, and likewise uniform compensation of the cornea's protrusion to be treated, the support body of a support element according to the invention should comprise a circumferential angle of at least 350° , in particular up to 360° .

Regardless of the respectively selected orientation of the flattening acting as a support surface, the support element according to the invention offers numerous possible variations while preserving its basic configuration according to the invention. For instance, the volumes occupied by the support element can be modified merely by parallel displacement of the flattening in the cross-sectional radial direction while preserving the diameter of the support body.

In order to increase the extent of peripheral reverse deformation of the cornea at certain positions and to reduce it at other positions, it is furthermore possible to provide, along the ring circumference of a support element according to the invention, local reductions or increases of the ring cross section, namely concerning the height in the cross-sectional radial direction or the width in the ring radial direction, and here concerning the outer as well as the inner diameter or only one of the two, or even both.

Accordingly, the local condition of the cornea, or the forces respectively to be applied locally, may be taken into account when configuring the support element according to the invention by varying the thickness of the support body along its circumference. To this end, the thickness of the support body may be varied in the direction of its height measured perpendicularly to the circle plane. As an alternative or in addition, to the same end, it is furthermore possible to vary the width of the support body measured parallel to the circle plane. In this case, it is of course not necessary to carry out the respective variation symmetrically with respect to the cross-sectional midpoint, but instead asymmetric indentations or protrusions may also be formed on the support element when this is necessary in view of the desired effect of the support element according to the invention in the implanted state. This configuration possibility provided by the invention may be crucial above all when correcting keratoconus phenomena lying off-centre to the support element.

An embodiment of the invention which is particularly gentle on the surroundings of the support element according to the invention is characterised in that the edge of the cross section of the support body, outside the region of the flattening, extends in the form of a circle arc.

In principle, it is conceivable for a front-side circumferential flattening formed on a support element according to the invention, which acts as a support surface in the implanted state, to be curved convexly or concavely inwards or outwards, respectively, while preserving its basic conical to horizontal alignment, when this is deemed expedient in view of the respectively desired effect. However, in taking into account the fact that the wall of the channel formed in the cornea contacting with the flattening in the implanted state will generally be designed to be planar, it is favourable if the flattening itself is likewise shaped to be planar. Consequently, according to another advantageous variant of the invention, the edge of the cross section of the support body extends in a straight line in the region of its flattening.

In order to prevent the cornea's regions adjacent to the edges of the support element from being stressed excessively in the implanted state, the transition between the flattening and the adjacent region of the support body should be designed to be rounded.

The handling of a support element according to the invention during insertion into the slit channel previously formed in the eye may be simplified by a flattened platform being formed on at least one end of its support body. This platform may serve as a contact point for the tool used by the surgeon. In order to facilitate secure coupling of the tool and the support body in this case, an opening may be formed in the flattened platform. The risk of injuring the cornea may be minimised by forming a chamfer in the region of the transition between the flattened platform and the support body.

A support element according to the invention may be produced from commercially available plastics which are highly biocompatible, from which the already known “intacs” are also made. Depending on the method of surgery employed and the instruments available, it may be expedient to use support elements having a support body formed in one piece or support elements whose support body is composed of more than one piece. Thus, also in a support element according to the invention, it is possible to assemble the support body, for example, from two half-rings.

The invention will be explained in more detail below with the aid of a drawing which represents exemplary embodiments. It is schematically shown in:

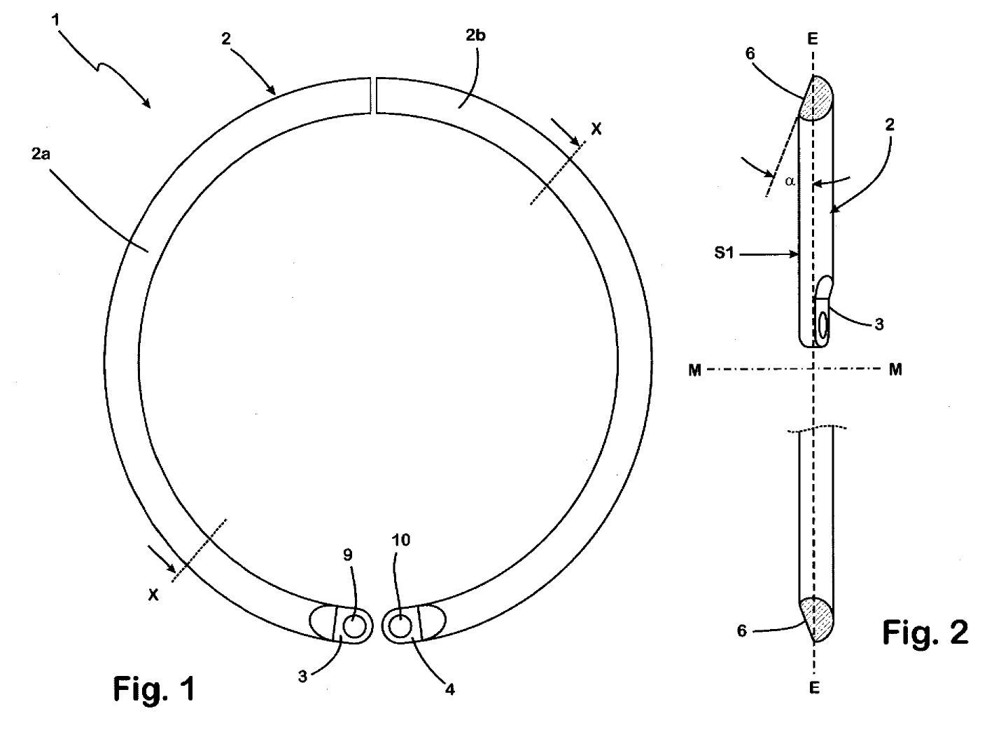

FIG. 1 a support element in a top view;

FIG. 2 the support element in a section along the section line X-X indicated in FIG. 1,

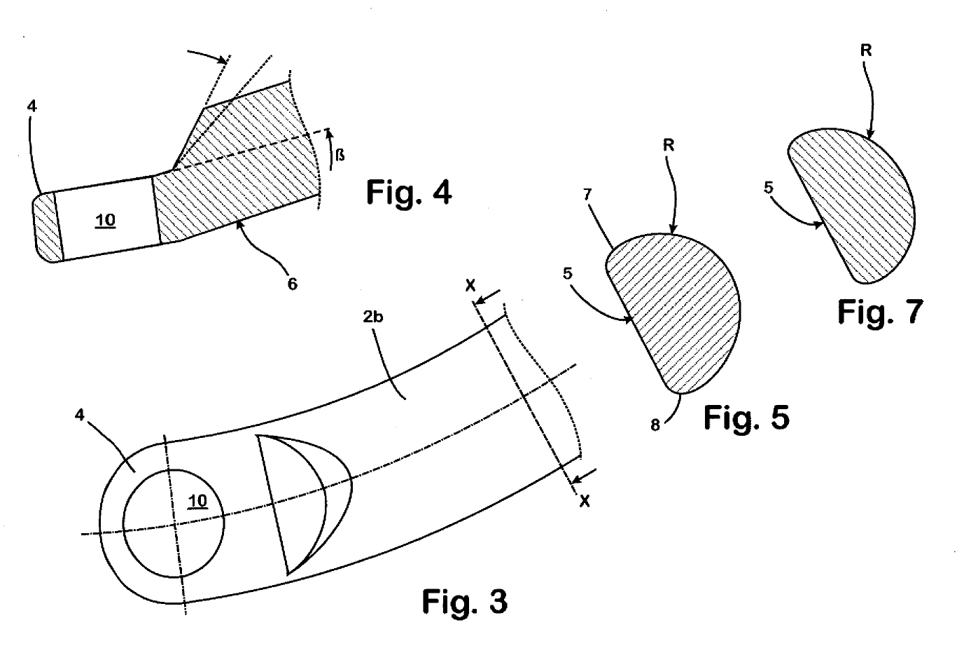

FIG. 3 an end section of the support element in an enlarged view corresponding to FIG. 1;

FIG. 4 the end section in a section along the ring axis of the support element;

FIG. 5 the support element in a section along the section line Y-Y indicated in FIG. 4;

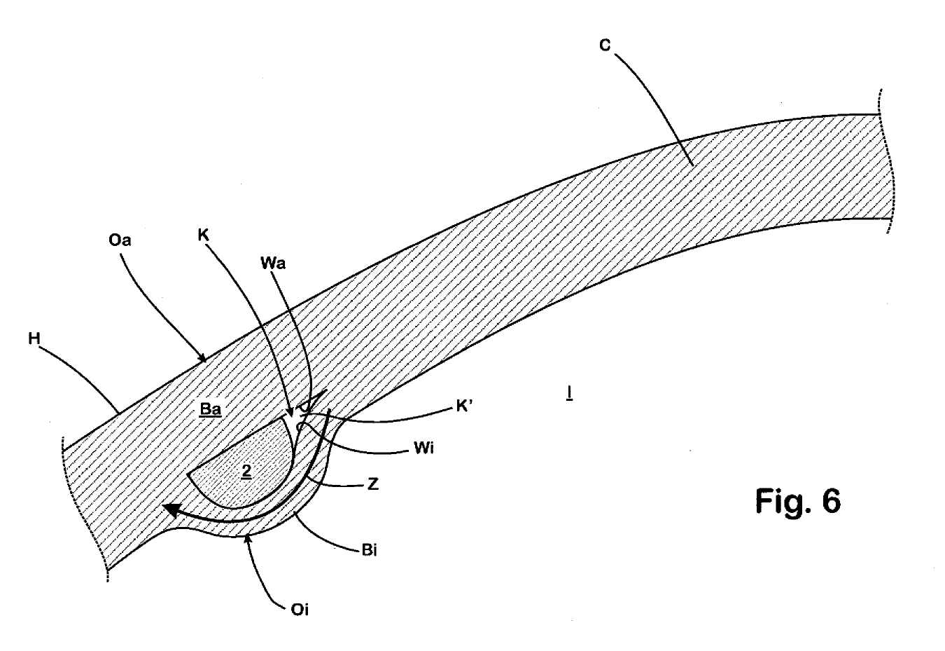

FIG. 6 a detail of a cornea of an eye with the support element implanted therein in a sectional view;

FIG. 7 a varied support element in a sectional view corresponding to FIG. 5.

The support element 1 shown in FIG. 1 comprises a support body 2 designed in a ring shape and covering an angle range of more than 350° , which is composed of two semi-ring shaped support body halves 2a, 2b on the end sections of which a flattened platform 3, 4 is respectively formed.

The support body 2 has a cross section whose basic shape corresponds to the shape of a circle, but which is obliquely flattened in the region 5 of its side assigned to a front side S1 of the support element 1. The edge R of the cross section extends in a straight-line profile in its flattened region, while it extends in the shape of a circle arc elsewhere. In this way, on the one front side S1 of the support body 2, a planar flattening 6 is formed which on the one hand encircles the support body 2 and on the other hand is inclined conically tapering in the direction of the mid-axis M of the support element 1 at an angle α relative to the circle plane E of the generally annular support element 1. The transitions 7, 8 between the flattening 6 and the adjacent sections of the support body 2 are designed to be rounded.

Through the positioning of the flattened region 5 of the cross section of the support body 2 the volume of the support body 2 can be adjusted with an otherwise unmodified geometry in a simple manner, as may readily be seen by comparing the variants shown in FIGS. 5 and 7.

The flattening 6 is extended beyond the free ends of the support body 2 to the free ends of the flattened platforms 3, 4 formed on the support body 2. The platforms 3, 4 have a thickness which is about half as great as the thickness of the support body 2. When introducing the support element 1 into a channel K incised in the cornea H of the eye to be treated, the platforms 3, 4 are used to hold the support element 1 by means of a suitable tool (not shown here) and to insert it into the channel K. In order to ensure secure holding, a through-hole 9, 10 aligned perpendicularly to the flattening 6 and passing through it is respectively formed in the platforms 3, 4.

In order to reduce the risk of inadvertent injury to a minimum when introducing the support element 1 into the channel K, the region of the transition U between the platforms 3, 4 and the support body 2 is respectively chamfered at an angle β of 45-60° .

In order to treat a keratoconus C (not shown here) on an eye (likewise not further represented here), the slit-shaped channel K is cut by means of a femtosecond laser known per se into the cornea H in a manner which is likewise known per se. The walls Wa, Wi delimiting this channel K extend essentially parallel to the curved surface Oa of the cornea H. The channel K is positioned so that the thickness Da of the corneal region Ba remaining between it and the outer surface Oa of the cornea H is greater than the thickness of the proximal region Bi remaining between it and the inner surface Oi of the cornea H.

Next, initially the first support body half 2a and then the second support body half 2b of the support element 1 are pushed into the channel K. The support body halves 2a, 2b, which together form the support body 2 of the support element 1, are respectively aligned so that its flattening 6 bears on the outer wall Wa assigned to the surface Oa of the cornea H.

Owing to its plane, conically aligned shape the flattening 6 bears tightly on the wall Wa without deforming it to a larger extent. At the same time, however, the proximal region Bi of the cornea H is displaced by the curved region of the support element 1 in the direction of the interior I of the eye, so that tensile forces Z directed outwards in the radial direction of the eye are created in this region. The effect of this is that the region of the cornea H, in which the keratoconus C has occurred, is pulled in the direction of the interior I of the eye until the curvature of the cornea H in the region of the keratoconus C is adapted again to the general curvature of the healthy region of the cornea H.

It should be noted that the free space K′ remaining at the side of the support element 1, or its respective support body 2, in the channel K has been represented exaggeratedly large in FIG. 6 for illustration. In practice the width of the channel K will of course be adapted to the width of the support element 1, or its support body 2, so that a free space K′ which is as small as possible remains at the side of the support element 1, or its support body 2, after implantation.

The embodiment of the invention as described in detail above is to be interpreted only as an example.

The basic shape selected for the support element according to the invention allows simple, low-resistance implantation and entails a very small risk of injury to the cornea owing to its shape being rounded on all sides. This does of course not exclude that—apart from the flattening always to be provided according to the invention—the cross section of a support element according to the invention can have cross-sectional shapes other than those explicitly mentioned here.

Advantageously, the inward-lying cross-section shape of a support element according to the invention will always be designed so that the detrimental effect on the corneal inner wall due to curvature and strain induced stresses is kept as small as possible.

It is also conceivable that in order to simplify the implantation process, instead of the platforms formed at the ends of the support body, a bore or a comparable opening is provided into which an implantation instrument can be fitted.

The precision of the channel configuration by the femtosecond laser also contributes to the high stability and reliability of the OP outcome. This precision relates both to the position of the channel relative to the corneal thickness and to the channel profile as an equidistant from the corneal contour. Such a slit channel means that each part, i.e. the distal (outer-lying) region and the proximal region (facing towards the body) has a thickness which is uniform per se.

When the support element according to the invention, with its flattening extending conically and serving as a support surface, is being implanted in the cornea, the outwardly facing channel wall offers an advantageous bearing and counter-support surface for the flattening of the support element. Owing to the large-area bearing, a unambiguously geometrically definable counter-bearing is obtained for the forces which result from the strain of the thinner, inner corneal layer through the support ring advantageously rounded, constructed inwards and perpendicularly to the corneal contour.

In addition to the diameters of the channel and support element, the extent of the corneal reverse deformation is determined by the size and shape of the cross-sectional area of the support element.

### REFERENCES

1 support element
2 support body
2a,2b support body halves
3,4 platform
5 flattened region of the cross section of the support body 2
6 planar flattening
7,8 transitions between the flattening 6 and the adjacent sections of the support body 2
9,10 through-openings
α angle
Ba,Bi corneal regions
β angle
C keratoconus
E circle plane of the support element 1
H cornea of the eye to be treated
I interior of the eye
K channel cut into the cornea H
K′ free space
M mid-axis of the support element 1
Oa,Oi surfaces of the cornea H
R edge of the cross section
S1 first front side of the support body 2
Wa,Wi walls delimiting the channel K
Z tensile forces

What is claimed is:

1-15. (canceled)

16. A support element to compensate for deformations of the cornea of an eye, having an annular support body in one or more pieces, characterised in that the support body, in a cross-sectional plane, has a cross section which is flattened on one side facing distally in an implanted state and otherwise rounded, so that a circumferential front-side flattening is formed on it.

17. A support element according to claim 16, characterised in that the flattening forms an annular disc surface tapering in the form of a cone.

18. A support element according to claim 16, characterised in that the flattening is inclined relative to a circle plane of the support element at a flattening angle ranging from 0° to 25° .

19. A support element according to claim 18, characterised in that the flattening angle is >0° .

20. A support element according to claim 16, characterised in that the support body comprises a circumferential angle of at least 350° .

21. A support element according to claim 16, characterised in that a thickness of the support body is varied along its circumference.

22. A support element according to claim 21, characterised in that the thickness of the support body is varied in the direction of its height measured perpendicularly to a circle plane.

23. A support element according to claim 21, characterised in that a width of the support body measured parallel to a circle plane is varied.

24. A support element according to claim 16, characterised in that an edge of the cross section of the support body extends in the form of a circle arc outside the region of the flattening.

25. A support element according to claim 16, characterised in that an edge of the cross section of the support body extends in a straight line in the region of its flattening.

26. A support element according to claim 16, characterised in that a transition between the flattening and an adjacent region of the support body is rounded.

27. A support element according to claim 16, characterised in that a flattened platform is formed on at least one end of the support body.

28. A support element according to claim 27, characterised in that an opening is formed in the flattened platform.

29. A support element according to claim 27, characterised in that a chamfer is formed in a region of a transition between the flattened platform and the support body.

30. A support element according to claim 16, characterised in that the support body is formed in one piece.

31. A support element according claim 16, characterised in that in the cross-sectional plane, the ratio of a cross-sectional area of the support body to the square of a length of the flattening of the support body is more than a value of about 0.65.

32. A support element according to claim 31, characterised in that in the cross-sectional plane, a ratio of the cross-sectional area of the support body to the square of the length of the flattening of the support body is less than a value of about 0.75.
